(19) United States
(12) Patent Application Publication
(10) Pub. No.: US 2023/0219203 A1
(43) Pub. Date: Jul. 13, 2023

(54) Insertion Instrument for Dental and Medical Device and Insertion Tool for Dental and Medical Device Comprising the Instrument

(71) Applicant: M3 HEALTH INDÚSTRIA E COMÉCIO DE PRODUTOS MÉDICOS, ODONTOLÓGICOS E CORRELATOS S.A., Jundiai (BR)

(72) Inventors: Alberto BLAY, São Paulo (BR); Alex Araújo NUCINI, Santa Rosa de Viterbo (BR); Jamil Awad SHIBLI, Guarulhos (BR); Samy TUNCHEL, São Paulo (BR)

(21) Appl. No.: 17/928,135

(22) PCT Filed: May 28, 2021
(86) PCT No.: PCT/BR2021/050229

§ 371 Date: Nov. 28, 2022

(30) Foreign Application Priority Data
May 28, 2020 (BR) BR102020010701-1

(51) Int. Cl. B25B 23/12; A61C 1/14; A61B 17/88; B25B 23/00

(52) U.S. Cl. CPC B25B 23/12; A61C 1/14; A61B 17/8875; B25B 23/0035

(57) ABSTRACT

The present invention discloses an instrument of medical/dental insertion, the instrument comprising: an axle; a fixing region; and an insertion element; where the fixing region makes the connection of the insertion element and the axle; the axis rotating the insertion element when connected; where the fastening region comprises a magnet configured to fix the insertion element on the axle.

Thus, one of the advantages of the present invention is to eliminate the natural wear that occurs in the retention, mechanical type, between the tools, besides facilitating the handling of instruments.

### FIELD OF INVENTION

The present invention relates in the field of tools, more precisely applied to tools for dental and orthopedic implants (medical indication) with magnetic force.

### BACKGROUND OF INVENTION

Ratchet-type keys are essential tools for ergonomics and agility and can be used in several areas. With them, injuries can be avoided by both effort and impact, because these tools allow the user to tighten a screw or nut repeatedly without always repositioning the tool, since the inner ratchet keeps the socket in place while the key arm is repositioned.

There are ratchet key models that may or may not be reversible and are marketed in the most commonly sizes in the socket fittings on the market (¼, ⅜, ½, ¾, among others). In addition to the fittings, there are the tips of the sockets that will come into contact with the nuts and screws that, in turn, are also available in wide variety in view of the diversity of screws and nuts with different types of fittings and sizes (hexagon, straited, torx, philips, allen, among others).

Currently the market offers a great diversity of accessories and accessories for the most diverse applications, being that these are usually sold in kits along with the ratchet keys. Much of these is produced in a special steel alloy that receives chromium and vanadium in its composition to increase durability and work resistance.

Additionally, it is common to use silicone o'rings with the ratchet wrench to promote the mechanical retention of a second wrench. In the field of dentistry, for example, this measure avoids direct contact of the user/professional with the key/instrument that will contact the patient's body during the surgical procedure. However, it should be considered that instruments of this nature need to be sterilized before each use. Thus, in the case of silicone rings, they are expected to gradually lose their properties due to the effect of high temperature during autoclaving process, so that they no longer firmly retain the insertion key. On the other hand, metal rings also lose their spring effect properties in view of natural wear related to time of use.

At the same time, it is possible to find on the market technologies that use magnets in magnetic tools or keys and that act by magnetic retention.

In this sense, patent document BR 10 2013 008127 2 describes a linear or rotational motion mechanism with magnetic memory retention. More specifically it comprises a mechanism for switching position and maintaining the position by the attraction between a magnet and a ferromagnetic core, with electricity consumption only during the switching of the position. The invention provides a linear or rotational motion mechanism with magnetic memory retention that only uses electrical energy to switch between the two positions and once one of the positions is established, there is no longer a need for electrical energy to maintain the position.

Document PI 0710446-4 describes a cylinder lock protection system that, when a magnet lock unlocks in response to a magnet approach, such a lock is unlocked. Thus, it can be switched between a state in which the insertion of a mechanical wrench into the key hole of an internal cylinder with pivotal rotation supported in a cylinder body is allowed and a state, and another in which the insertion of the mechanical key into the key hole is prevented, so that a shutter mechanism is arranged in the cylinder body, the shutter mechanism having a pivotal rotation member and a shutter plate, the pivoting limb having an insertion hole to allow the insertion of the mechanical key into the key hole and pivotal rotation around the same axis. In this way, the pivot axis of the inner cylinder can be moved in response to a pivoting operation when the magnet lock is unlocked and the shutter plate being operated to the pivoting limb so as to move between an open position in which the insertion hole is opened and a closed position in which the insertion hole is fully closed in response to pivoting of the pivoting limb. In addition to the first aspect, the magnet is provided in a key box that attaches prominently with the pivoting member and is able to pivot the pivot member in the united state, the magnet enabling the magnet lock to be unlocked in a state where the key box is attached with the pivoting member, in which the key box retracts the mechanical key, so that the mechanical key is made to project itself from the key box and be inserted into the key hole in a state in which the pivoting limb is pivoted, so as to place the shutter plate in the open position, and a coupling part is provided on the cylinder body side, the coupling part preventing the key box from becoming detached from the pivoting limb by coupling with the key box when the mechanical key is made to protrude from the key box at least after the magnet latch is unlocked and the shutter plate is placed in the open position.

Document MU9000900-2 refers to a construction arrangement introduced in rapid coupling acting by magnetic action and which is intended for the retention of shelf supports in bushings fixed to vertical panels, partitions, furniture or walls, in particular for display of goods. The proposed coupling comprises a rod that penetrates inside a bushing, which is fixed to the vertical panel, whose movement is rectilinear, without inclinations or rotations. The rod has any prismatic end, optionally heist, that penetrates into an inner chamber of the bushing with the same cross section, whose bottom has a magnet that retains the rod. The bushing has a protruding edge and an outer thread that receives a fastening nut on the vertical panel. Optionally, the bushing attachment nut on the panel may also have a protruding edge. Also optionally, both the protruding edge of the bushing and the nut can feature a peripheral groove that houses an o-ring for better grip on the panel surface.

The document CN201879855 belongs to the technical field of medical device, in particular a magnetic retainer for teeth, which uses a permanent magnet of neodymium-iron-boron of rare earths to generate a strong magnetic attraction force and is used for the retention of the dental prosthesis.

In view of the above, it is understood that the techniques understood by the state of the art have advantages; however, they require continuous improvements.

In this sense, this patent application presents a solution that combines technical differentials, economic advantages, safety and reliability in surgical procedures that will be revealed below.

### OBJECTIVES OF INVENTION

Thus, it is an objective of the present invention to provide a solution to the challenges and limitations listed above, presenting a dental and medical insertion tool that promotes less wear between the insertion element and the axle, using a magnetic fitting.

It is an objective of the present invention to provide an instrument with innovative design, which retains the element of insertion in precise fitting way, promoting safety and reliability.

It is also an objective of the present invention to provide an autoclavable tool with protections for magnetic elements, in order to avoid corrosion.

### SUMMARY OF INVENTION

The present invention achieves this and other objectives through an instrument of medical/dental insertion, the instrument comprising: an axle; a fixing region; and an insertion element; where the fixing region makes the connection of the insertion element and the axle; the axle rotating the insertion element when connected; wherein the fixing region comprises a magnet configured to fix the insertion element on the axle.

According to a preferred configuration, the fastening region comprises at least one hole in a position perpendicular to the axle, wherein the magnet is inserted into the hole in order to fix the insertion element on the axle, and the magnet is preferably manufactured in samarium, cobalt or neodymium, and may comprise an external layer of moisture-resistant protection.

Preferably, the insertion element is configured to connect to a medical/dental device, where the shaft is manufactured in Stainless Steel according to ASTM F-899, the length of the medical/dental insertion instrument varies between 16 mm and 250 mm, preferably from 16 mm to 40 mm for dental applications and between 150 mm and 250 mm for orthopedic keys and the magnet fitting hole has a width between 2.0 mm and 8.00 mm.

The present invention also describes a dental insertion tool, comprising a medical/dental insertion instrument according to the above defined, in which the tool is of the type against angle and/or ratchet.

### DETAILED DESCRIPTION OF THE DRAWINGS

The present invention will be more detailed and elucidated based on the following figures:

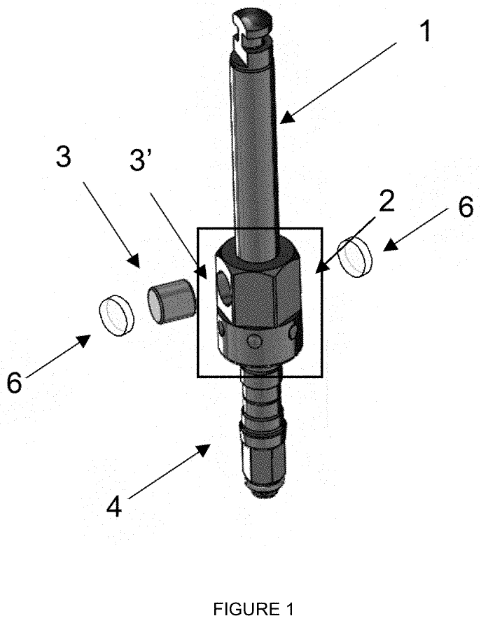

FIG. 1 illustrates an instrument of dental and medical insertion according to a preferred embodiment of the present invention;

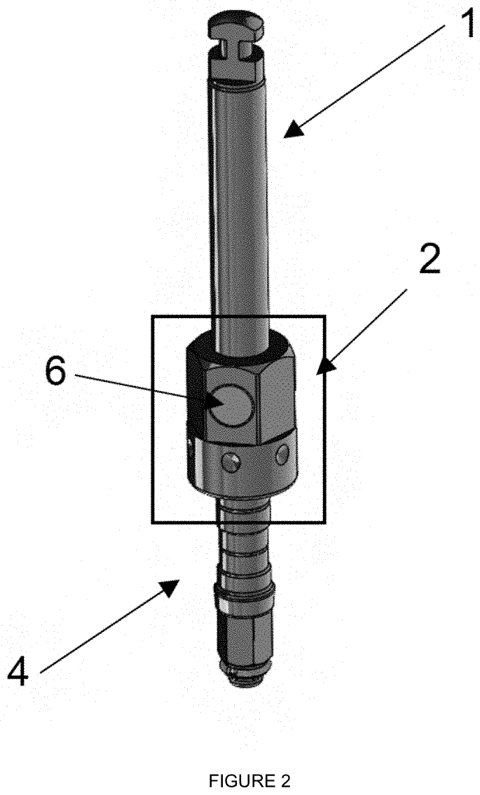

FIG. 2 shows an instrument of dental and medical insertion according to a preferred embodiment of the present invention;

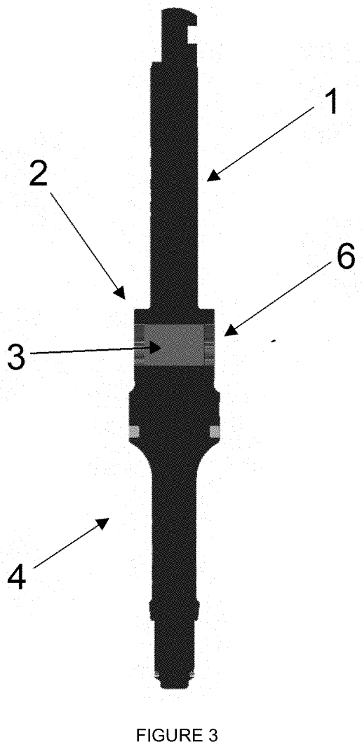

FIG. 3 shows the cut-off view of a dental and medical insertion instrument according to a preferred embodiment of the present invention; and

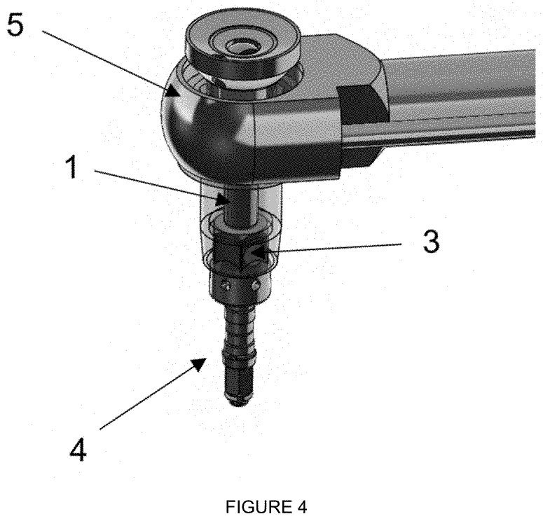

FIG. 4 illustrates a dental and medical insertion tool comprising a dental and medical insertion instrument according to a preferred embodiment of the present invention.

### DETAILED DESCRIPTION OF THE INVENTION

Regarding, FIGS. 1 and 2, present invention describes an instrument of medical/dental insertion, the instrument comprising: an axle 1; a fixing region 2; and an insertion element 4; where the fixing region 2 makes the connection of the insertion element 4 and the axle 1; the axle 1 rotating the insertion element 4 when connected; where the fixing region 2 comprises a magnet 3 configured to fix the insertion element 4 on axle 1.

According to a preferred embodiment, the fixing region 2 comprises at least one hole 3′ in a position perpendicular to axle 1, wherein the magnet 3 is inserted into hole 3′ in order to fix the insertion element 4 on axle 1, and magnet 3 is preferably manufactured in samarium, cobalt or neodymium, and may comprise an external layer of humidity-resistant protection, as shown in the cut-view of FIG. 3.

Preferably, the insertion element 4 is configured to connect to a medical/dental device, where shaft 1 is manufactured in Stainless Steel according to ASTM F-899, the length of the dental and medical insertion instrument varies between 16 mm and 250 mm, and the 3′ hole for magnet fitting 3 comprises a width between 2.0 mm and 8.0 mm.

Still in reference to the FIG. 3, the 3′ hole can receive a cover 6 on both sides, which prevents the magnet from being exposed to the environment or humidity at the time of autoclaving, preventing the magnet from being corroded.

The cover 6 can be made of resin or polymers, so as to seal the hole 3′ with the magnet inside, being ideal for autoclave sterilization processes.

The present invention also describes a tool 5 of dental and medical insertion, comprising an instrument of dental and medical insertion according to the above defined, in which tool 5 is of the type against angle and/or ratchet, as shown in FIG. 4.

Given the description above, one of the advantages of the present invention is to eliminate the natural wear that occurs in retention, mechanical type, between tools, while also facilitating the handling of instruments.

### Device Operation

The axle 1 is tightly fitted to the tool 5, as shown in FIG. 4. Thus, when the torque is applied by the professional in tool 5, the same torque is applied to axis 1, which in turn transmits the torque to the insert 4, which finally transmits the torque to the implant or prosthesis screw.

Due to the fact the insertion element 3 is fixed, by magnetic force of the magnet 3 present in the hole 3′ of the fixation region 2 of the axis 1, this region has little wear compared to the traditional fitting, in which relative movement between the pieces in the fixing region causes small collisions that over time increases the wear of parts in that region.

Another advantage is the durability of its components, which are exposed multiple times to processes such as autoclave.

Having described an example of a preferred embodiment of the present invention, it should be understood that the scope of the present invention covers other possible variations of the inventive concept described, being limited only by the content of the claims, including the possible equivalents.

1. Instrument of medical/dental insertion, the instrument comprising:
an axle (1);
a fixing region (2); and
an insertion element (4);
wherein the fixing region (2) makes a connection of the insertion element (4) and the axle (1);
the axle (1) rotating the insertion element (4) when connected;
characterized by the fact the fixing region (2) comprises a magnet (3) configured to fix the insertion element (4) on the axle (1) by means of magnetic force.

2. Instrument, according to claim 1, characterized by the fact the fixing region (2) comprises at least one hole (3′) in a position perpendicular to the axle (1), wherein the magnet (3) is inserted into the hole (3′) in order to fix the insertion element (4) to the axle (1).

3. Instrument, according to claims 1 and 2, characterized by the fact the magnet (3) comprises one or more covers (6) made of material selected from polymer, resin or anticorrosion film.

4. Instrument, according to claim 1, characterized by the magnet (3) being manufactured from material selected from samarium, cobalt, neodymium, and combinations between them.

5. Instrument, according to claim 1, characterized by the fact the insertion element (4) being configured in order to connect to a medical/dental device.

6. Instrument, according to claim 1, characterized by the fact the shaft (1) being made of Stainless Steel.

7. Instrument, according to claim 1, characterized by the fact the length of the insertion instrument being for dental application vary between 16 mm and 40 mm.

8. Instrument, according to claim 1, characterized by the fact the length of the insertion instrument being for medical/orthopedic application vary between 150 mm and 250 mm.

9. Instrument, according to claim 1, characterized by the fact the hole (3′) for magnet fitting (3) has a width between 3.0 mm and 5.0 mm.

10. Tool (5) of medical/dental insertion, characterized by the fact it comprises an instrument of medical/dental insertion as defined in claim 1.

11. Tool (5) of medical/dental insertion, according to claim 10, characterized by the fact it is a contra-angle type tool and/or ratchet type tool.

12. Instrument, according to claim 1, characterized by the fact the covers (6) are configured to fully seal up the at least one hole (3′), such it prevents the magnet from being exposed to the environment or humidity during autoclaving processes.
